(19) United States
(12) Patent Application Publication
(10) Pub. No.: US 2026/0233221 A1
(43) Pub. Date: Aug. 13, 2026

(54) MICROFLUIDIC DEVICE FOR USE IN A SYSTEM FOR MEASURING AT LEAST ONE MECHANICAL PROPERTY OF AT LEAST ONE OOCYTE

(71) Applicants: COLLEGE DE FRANCE, Paris (FR); UNIVERSITE D'EVRY VAL D'ESSONNE, Evry (FR); INSTITUT NATIONAL DE LA SANTE ET DE LA RECHERCHE MEDICALE (INSERM), Paris (FR); CENTRE NATIONAL DE LA RECHERCHE SCIENTIFIQUE, Paris (FR)

(72) Inventors: Lucie BARBIER, Paris (FR); Rose BULTEAU, Evry (FR); Clément CAMPILLO, Evry (FR); Marie-Emilie TERRET, Paris (FR); Marie-Hélène VERLHAC, Paris (FR)

(73) Assignees: COLLEGE DE FRANCE, Paris (FR); UNIVERSITE D'EVRY VAL D'ESSONNE, Evry (FR); INSTITUT NATIONAL DE LA SANTE ET DE LA RECHERCHE MEDICALE (INSERM), Paris (FR); CENTRE NATIONAL DE LA RECHERCHE SCIENTIFIQUE, Paris (FR)

(21) Appl. No.: 19/471,148

(22) PCT Filed: Apr. 3, 2024
(86) PCT No.: PCT/EP2024/059100

§ 371 Date: Sep. 30, 2025

(30) Foreign Application Priority Data
Apr. 3, 2023 (EP) 23166408.7

(51) Int. Cl. B01L 3/00; C12N 5/075; G01N 15/10; G01N 15/14; G01N 15/1433

(52) U.S. Cl. CPC B01L 3/502715; B01L 3/502746; C12N 5/0609; G01N 15/1433; G01N 15/147; G01N 15/1484; B01L 2200/0663; B01L 2300/0816; B01L 2400/0487; G01N 2015/1006; G01N 2015/1495

(57) ABSTRACT

A microfluidic device for use in a system for measuring at least one biomarker, of at least one oocyte, the microfluidic device including at least one microfluidic channel configured for a passage of a flow of fluid with at least one oocyte, the microfluidic channel including a restriction section configured to deform the oocyte in at least a first direction and a second direction perpendicular to a microfluidic channel axis, the first direction being different from the second direction.

### FIELD

The present invention relates to a microfluidic device for use in a system for measuring at least one biomarker of at least one oocyte. The present invention also relates to the associated system and method for measuring at least one biomarker of at least one oocyte.

### BACKGROUND

Oocyte morphogenesis is error-prone. The number of poor-quality oocytes increases with maternal age, especially after 35 years, leading to a higher risk of infertility, miscarriage, and congenital syndromes, such as Down syndrome.

The age-related decline in oocyte quality is a major societal problem, as the average maternal age gradually increases worldwide. Indeed, the average maternal age was 26.5 years old in the 80's compared to 30.2 years old in 2020. Consequently, the use of Assisted Reproductive Technology (ART) is increasing in Europe. Indeed, it has increased by 7% per year since 2000. Yet, human ART success rates are low, with only 20% of ART cycles resulting in a live birth. This low success rate represents a significant burden due to the heavy treatment and high risk of miscarriage.

In current fertility treatments, 2 to 20 mature oocytes per patient are collected after hormonal stimulation. One option is to freeze the oocytes for fertility preservation. Another option is to fertilize them, producing one-cell embryos called zygotes. A zygote corresponds to the cell formed after fusion of an ovum and a spermatozoid, before its first division. As spermatozoids volume is infinitesimal compared to the oocyte, the zygote is therefore the same size as the oocyte.
Embryo: includes the zygote and all subsequent stages of development up to 8 weeks in humans.

Before transfer to the patient, embryologists seek embryos with the highest quality to maximize chances of healthy birth, while avoiding multiple pregnancies. Typically, in in-vitro fertilization (IVF) procedures, oocyte/spermatozoid fusion takes place in vitro and the resulting zygote is cultured for between two to five days before re-implantation in a uterus. From day five the embryo is called a blastocyst, containing around 200 cells and still the same size as the oocyte or zygote.

Blastocyst quality can be predicted from the viscoelastic properties of the oocyte or zygote in the hours following fertilization. For instance, extra-stiff or extra-soft zygotes produce fewer viable blastocysts in human and mice, and result in fewer pups in mice. As another example, in mouse oocytes and zygotes, the tension generated by the cortical acto-myosin underneath the cell membrane (i.e. cortical tension) is tightly regulated to ensure proper development. Extra-soft oocytes present defects in chromosome alignment, resulting in abnormal chromosome numbers, which is associated with miscarriage and congenital syndromes.

In this context, oocyte mechanical properties represent readily available quantitative parameters that reflect oocyte quality and therefore a promising biomarker for oocyte selection in assisted reproduction. However, current mechanical measurement methods were developed for research purposes and are therefore not easily applicable in the medical procedures of ART.

The mechanical characterization of an object involves the measurement of force-deformation reaction curves. Oocytes are spherical multilayer-viscoelastic objects with internal pressure and surrounded by a glycoprotein layer with a typical size around 100 μm in diameter. Oocytes sizes in mammals can vary from 50 to 3000 μm, but most of the cases has an oocyte range from 60 to 150 μm. According know examples known studies has documented that oocyte size is not related to the animal size: platypus—2.5 mm, Dasyurus—240 μm, Didelphis—150 μm, Armadillo—80 μm, whales—140 μm, talpa—125 μm, hedgehog—100 μm, mouse—70 to 75 μm, Rat—70 to 75 μm, Guinea pig—75 to 85 μm, rabbit—120 to 130 μm, dog—135 to 145 μm, Cat—120 to 130 μm, horses—135 μm, Sheep—120 μm, Goat—140 μm, Pig—120 to 140 μm, Cattle—120 μm, bat—95 to 105 μm, Primates such as Gibbon, Rhesus Monkey, Gorilla 110 to 140 μm, Panda—140 μm, White rhinoceros—100 μm. Because of these physical properties, current methods for studying the mechanical properties of oocytes involve extensive micromanipulation such as micropipette aspiration or atomic force microscopy (AFM). These methods are appropriate for research purposes but they cannot be implemented at larger scales for various medical applications. Same constrains apply for zygote and early blastocysts prior re-implantation as their size remains unchanged from oocyte stage to the day of reimplantation. Thus, the present invention can also be considered as a measurement tool for every stage of the in vitro procedure: from oocyte collection to reimplantation of the embryo in the uterus. In the context of the invention an ooctyte is either a not fertilized oocyte or fertilized oocyte, also called a zygote.

In microfluidic devices, objects can undergo controlled micrometer-scale deformation in well-defined geometries and flow. Microfluidics has been used in translational research to measure single cell mechanical properties as diagnostic biomarkers for diseases such as malaria, chronic inflammation, and cancer progression. Yet, current microfluidic devices are ideal for measuring small cells, with sizes inferior to 50 μm, in large volumes such as blood samples, but not for large and rare objects such as oocytes that have a size around 100 μm and neither for detailed mechanistic analysis before and after oocyte fertilization.

A few microfluidic approaches have been reported for the automated manipulation of oocytes and embryos in ART routines such as oocyte denudation, on-chip fertilization, and embryo culture. However, none of them have successfully demonstrated the implementation of mechanical oocyte measurement for quality assessment in a microfluidic device.

Therefore, the problem solved by the invention is to provide a cost-effective, easy-to-use and non-invasive device to measure the mechanical properties of an oocyte to determine its quality prior to cryopreservation, in vitro maturation and/or in vitro fertilization that may be implemented at larger scales. The problem solved by this invention brings solution for in-vitro fertilization for Human, but also for animal breeding, as well as the use of IVF for conservation of endangered animal species.

### SUMMARY

To solve this problem, the invention relates to a microfluidic device for use in a system for measuring at least one biomarker of at least one oocyte. The microfluidic device comprises at least one microfluidic channel configured for a passage of a flow of fluid comprising at least one oocyte. The microfluidic channel also comprises a restriction section configured to deform the oocyte in at least a first direction and second direction perpendicular to a microfluidic channel axis, the first direction and the second direction being non-aligned.

In other words, the microfluidic device of the invention is a cost-effective easily scalable device that allows to capture the deformations of an oocyte through a restriction section with controlled dimensions. By constricting the oocyte along at least two dimensions of space, it is possible to better control the flow inside the restriction section compared to one-dimensional restriction sections. Indeed, a restriction in at least two non-aligned dimensions of space prevents fluids from passing through gaps between the trapped oocyte and the microchannel walls, as observed with one-dimensional restriction sections. Thus, the oocyte is not exposed to shear stress during deformation and is not damaged in the process. Indeed, experiments have shown that there is no noticeable morphological alteration neither spindle damage in the oocyte after passage into the microfluidic device of the invention. Advantageously, the microfluidic device may be adapted to any mammalian oocyte. It may be used for human ART but also to improve breeding yields in livestock. In any embodiment of the invention, an oocyte can be a not fertilized oocyte, or a fertilized oocyte also called zygote.

Advantageously, the oocyte has an oocyte size and the restriction section has a transversal section comprised between 70% and 90% of the oocyte size, preferably between 75% and 85% of the oocyte size, and more preferably of 80% of the oocyte size.

The transversal section is defined to minimize oocyte deformation while ensuring accuracy of measurement. The transversal section is advantageously adapted according to the physical characteristics of the oocyte of the species studied. AFM, micropipette measurements, mechanical modeling or numerical simulation may be used to determine the most optimized transversal section. The transversal section may correspond to the cross-section of the restriction section.

According to an embodiment, the restriction along the first direction is identical to the restriction along the second direction. In practice, the microfluidic channel has a polygonal-shaped cross-section.

Indeed, a polygonal-shaped cross-section allows a better imaging of the oocyte and a better control over the oocyte deformation, while reducing the risks of damaging the oocyte.

According to an embodiment, the restriction section comprises a section of minimal dimensions.

More particularly, the restriction section successively comprises:
a first ramped section configured to gradually constrict the oocyte,
a section of minimal dimensions, and
a second ramped section configured to gradually release restriction on the oocyte.

Ramped segments allow a size transition between the enlarged section upstream the restriction section and the segment with minimal size restriction. Therefore, it allows a progressive deformation of the oocyte with limited mechanical stress. Moreover, it facilitates the measurement of the at least one biomarker.

According to an embodiment, the microfluidic channel has an enlarged section upstream the restriction section and a transverse dimension of the enlarged section is preferably comprised between 200% and 300% of the oocyte size. The transverse dimension may correspond to the cross-section of the enlarged section.

According to another embodiment, the ratio between a length of the section of minimal dimensions and a length of the ramped sized along the microfluidic channel axis is comprised between 0.3 and 1.

Advantageously, the section of minimal dimensions has a length comprised between 1 and 3 time the ratio between the oocyte size and a transverse dimension of the section of minimal dimensions.

The smaller the transverse dimension of the section of minimal dimensions is, the longer the length of the section of minimal dimensions should be. Indeed, the oocyte should preferably be completely deformed to better measure the oocyte at least one biomarker.

Additionally, the ratio between a length defined between the oocyte inlet and the restriction section and a length of the section of minimal dimensions may be comprises between 35 and 45.

Advantageously, the restriction section may comprise at least one transversal dimension that is variable. In other words, the restriction section may comprise at least one wall that is deformable along at least one of the first direction and second direction This allows to induce variation in the constriction section transversal dimensions, thus allowing to perform measurements on oocytes from different species using the same design of microfluidic device. Advantageously said first and second direction being orthogonal directions.

According to an embodiment, the microfluidic device may comprise:
a flow inlet configured to be connected to a pressure controller,
a flow outlet,
an oocyte inlet configured for the introduction of an oocyte, and
an oocyte outlet configured for the extraction of the oocyte,
   the oocyte inlet being positioned between the flow inlet and the restriction section and the oocyte outlet being positioned between the restriction section and the flow outlet.

According to another aspect, the invention relates to a system for measuring at least one biomarker of at least one oocyte comprising:
a microfluidic device such as described above,
a pressure controller configured for applying a pressure on the oocyte to transport the oocyte through the restriction section,
an imaging device configured for acquiring at least one image of the oocyte during its passage into the restriction section, and
at least one processor configured to process the at least one image to measure the at least one biomarker.

For instance, the processor may be configured to determine a position of the oocyte. Such position may be used to determine a curve of the position of the oocyte in the restriction section as a function of time and/or applied pressure. Surface curvature and pressure required to drive the oocyte at different positions of the restriction segment are linked to oocyte cortical tension.

As another example, the processor may be configured to determine a strain of the oocyte. Strain may be defined as a fraction of oocyte length extension, aspect ratio, circularity or any other quantitative shape parameter. Maximal strain and strain evolution in the function of time and/or applied pressure are linked to oocyte viscoelastic properties.

According to an embodiment, the system further comprises a pressure sensor configured to measure the pressure applied inside the microfluidic channel, at least one processor being configured to control the pressure controller to adapt the pressure based on at least one measurement of the pressure sensor.

According to an embodiment, the system further comprises a flow sensor configured to measure the flow inside the microfluidic channel, the at least one processor being configured to control the pressure controller to adapt the flow in the microfluidic device based on at least one measurement of the flow sensor.

According to another aspect, the invention relates to a method for measuring at least one biomarker of at least one oocyte using a system such as described above, comprising:
applying a pressure into the microfluidic device, to transport at least one oocyte toward the restriction section,
acquiring at least one image of the oocyte during its passage into the restriction section, and
processing the at least one image to measure the at least one biomarker.

### BRIEF DESCRIPTION OF THE DRAWINGS

FIG. 1 is a perspective view of a microfluidic device according to the invention,

FIG. 2 is a perspective view of the restriction section from FIG. 1,

FIG. 3 is a side-view of the restriction section from FIG. 2,

FIG. 4 is a top-view of the restriction section from FIG. 2,

FIG. 5 is a cross-section of the microchannel showing a pillar according to the invention,

FIG. 6 is a schematic representation of the system according to the invention, including a microfluidic device from FIG. 1,

FIG. 7 is a bloc diagram of the method according to the invention,

FIG. 8 is a set of images captured by the imaging device from the system of FIG. 6 and a set of images processed by the processor from FIG. 6,

FIG. 9 is a curve of the position of the oocyte in the restriction section as a function of time and pressure,

FIG. 10 is a curve of the length elongation of the oocyte as a function of time,

FIG. 11 is a curve of the pressure and flow as a function of time,

FIG. 12 is a formula linking the length elongation with the width of the oocyte as measured during its passage through the restriction section, and

FIG. 13 is a curve of the aspect ratio as a function of the pressure.

### DETAILED DESCRIPTION

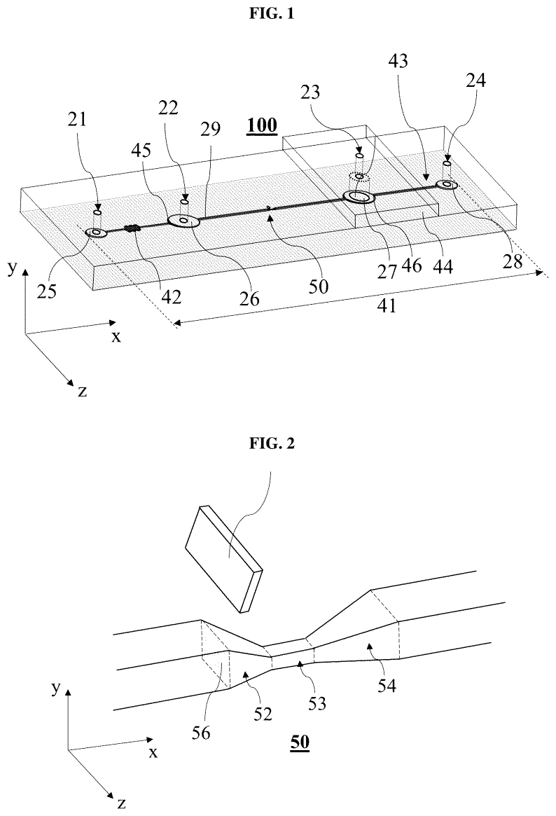

The present disclosure will be described in reference to different embodiments of a microfluidic device 100 for measuring at least one mechanical property of at least one oocyte, as illustrated on FIG. 1.

The microfluidic device 100 may have a parallelepipedal shape with a length comprised between 30 mm and 100 mm, a width comprised between 5 and 50 mm and a thickness comprised between 2 and 20 mm.

The microfluidic device 100 may be made with any transparent and bio-compatible material compatible with fabrication of microfluidic chips such as glass, silicon or a polymer like the Polydimethylsiloxane (PDMS).

At least one microchannel 43 may be etched or molded into the bio-compatible material. Advantageously, several parallel and independent microchannels 43 may be etched or molded into the bio-compatible material, thus allowing a parallel and simultaneous treatment of several oocytes.

The microfluidic device 100 may be molded using a photolithography protocol. The protocol comprises steps of exposure of a photosensitive resin covered by a mask in the shape of the microchannel 43 by an UV light to solidify the resin. Alternatively, the mold may be obtained by brass micro-milling, or 3D printing.

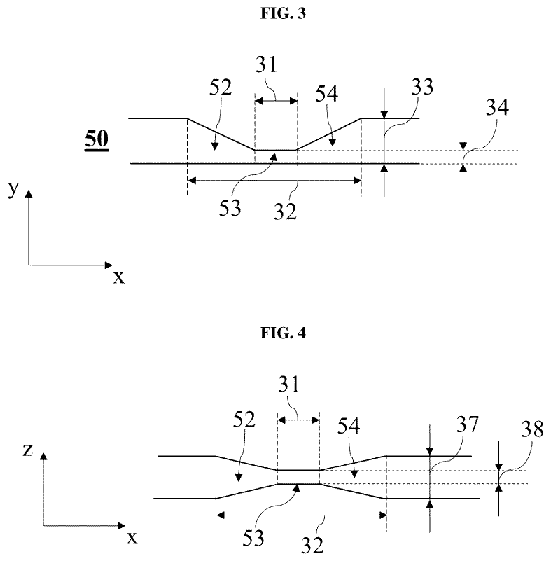

The microchannel 43 has a total length 41 comprised between 20 mm and 60 mm, preferably 40 mm and a polygonal-shaped cross-section 56 such as illustrated in FIG. 2. The cross-section 56 may be a rectangle, a square, a pentagon, a hexagon and even a circle. As illustrated in FIGS. 3 and 4, outside of the restriction section 50, the height 33 of the cross-section 56 is comprised between 0.15 and 0.25 mm, preferably 0.2 mm and the width 37 is comprised between 0.15 and 0.25 mm, preferably 0.2 mm.

The microfluidic device 100 includes a flow inlet 21 that connects the upper surface of the microfluidic device 100 with the microchannel 43 and more precisely connects the upper surface of the microfluidic device 100 with a first entrance 25 of the microchannel 43. The flow inlet 21 may be positioned at one extremity of the microchannel 43. The flow inlet 21 may be a cylinder-shaped opening with a diameter comprised between 0.2 and 0.5 mm, preferably 0.35 mm. The flow inlet 21 is configured for connection with flexible tubing. The first entrance 25 is preferably circular-shaped to avoid aggregation of debris in angles. It has a diameter comprised between 0.5 and 2 mm, preferably 1 mm. The first entrance 25 acts as a pool of fluid to ensure that a laminar flux is generated inside the microchannel 43.

The microfluidic device 100 also includes a flow outlet 24 that connects the upper surface of the microfluidic device 100 with the microchannel 43 and more precisely connects the upper surface of the microfluidic device 100 with a first exit 28 of the microchannel 43. The flow outlet 24 may be positioned at the opposite extremity of the microchannel 43. The flow outlet 24 may be a cylinder-shaped opening with a diameter comprised between 0.2 and 0.5 mm, preferably 0.35 mm. The flow outlet 24 is configured for connection with flexible tubing. The first exit 28 is preferably circular-shaped to avoid aggregation of debris in angles. It has a diameter comprised between 0.5 and 2 mm, preferably 1 mm. The first exit 28 acts as a pool of fluid to ensure that a laminar flux is generated inside the microchannel 43.

The microfluidic device 100 also includes an oocyte inlet 22 that connects the upper surface of the microfluidic device 100 with the microchannel 43 and more precisely connects the upper surface of the microfluidic device 100 with an oocyte entrance 26 of the microchannel 43. The oocyte inlet 22 may be positioned after the flow inlet 21 along the x-axis of the microchannel 43. The oocyte inlet 22 may be a cylinder-shaped opening with a diameter comprised between 0.2 and 0.5 mm, preferably 0.35 mm. The size of the oocyte inlet 22 allows oocyte deposition in the device with standard pipettes used for ART. Size-ratio between the diameter of the inlet 22 and the diameter of the oocyte entrance 26 limits flow when closing the device and avoids risk of flushing the oocyte. The oocyte entrance 26 is preferably circular-shaped to avoid aggregation of debris in angles. It has a diameter comprised between 1 and 4 mm, preferably 2 mm. The oocyte entrance 26 acts as a pool of fluid to avoid the oocyte going straight into the restriction section 50. The ratio between the diameter of the first entrance 25 and a transversal dimension of the portion 29 of the microchannel 43 that exits the first entrance 25 is preferably comprised between 8 and 12.

The portion 29 of the microchannel 43 that exits the first entrance 25 and links the first entrance 25 to the restriction section 50 has a length of at least two times the first entrance 25 diameter, that is at least 2 mm. The length of the portion 29 is chosen to allow good sedimentation and good separation between the objects that transit through the microchannel 43. Indeed, sometimes debris are introduced at the same time as the oocyte in the microfluidic device 100. The smaller debris will transit faster toward the restriction section 50 while bigger debris may either be trapped into the first entrance 25 or transit much slower than the oocyte toward the restriction section 50.

The microfluidic device 100 also includes an oocyte outlet 23. Advantageously, the microfluidic device 100 includes an upper layer 44 with a width comprised between 5 and 50 mm. The oocyte outlet 23 connects the upper surface of the microfluidic device 100 with the microchannel 43 and crosses through the upper layer 44. The oocyte outlet 23 is a cylinder-shaped opening with a diameter comprised between 0.2 and 0.5 mm, preferably 0.35 mm. More precisely, the oocyte outlet 23 is connected to an oocyte exit 27. The oocyte exit 27 is preferably circular-shaped to avoid aggregation of debris in angles. It has a diameter comprised between 1 and 4 mm, preferably 2 mm. The oocyte exit 27 has a height comprised between 1 and 4 mm, preferably 2 mm. It acts as a large reservoir to ensure oocyte arrest, with a smaller oocyte outlet 23 to recover oocyte with standard pipettes used for ART. Size-ratio between the oocyte outlet 23 and the oocyte exit 27 limits flow when closing the device and avoids risk of flushing the oocyte.

Advantageously, the microfluidic device 100 also includes a portion with resistance properties 42 with a cross section of reduced dimensions. For instance, the dimensions of the portion with resistance properties 42 may be reduced along a given direction. The length along this direction may be comprised between 0.3 and 0.7 mm. This portion with resistance properties 42 allows a precise flow control in the microfluidic device 100 and more precisely, it ensures a laminar flux inside the microchannel 43.

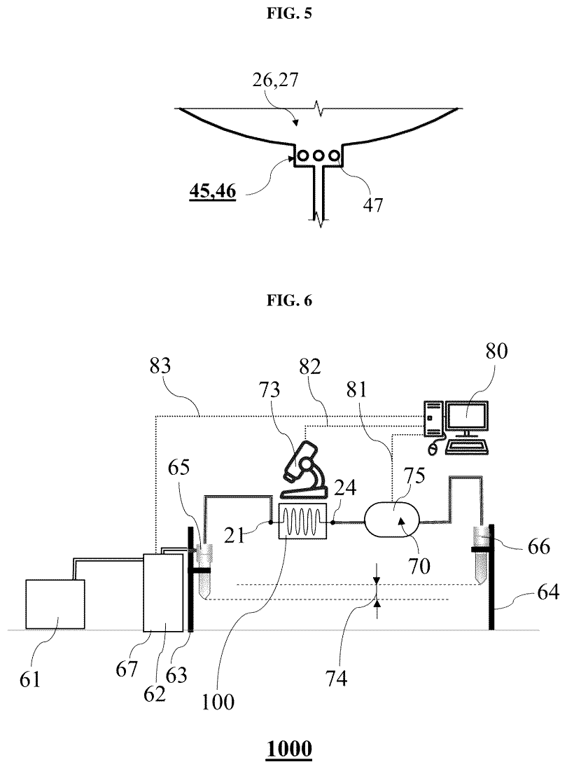

Advantageously, the microfluidic device 100 also includes pillars 45, 46 such as illustrated in FIG. 5. The first pillar 45 is a transversal barrier localized in the section of the microchannel 43 that links the first entrance 25 to the oocyte entrance 26. The first pillar 45 comprises at least one opening 47 configured for the passage of a fluid but configured to block the passage of the oocyte. The second pillar 46 is also a transversal barrier localized in the section of the microchannel 43 that links the oocyte exit 27 to the first exit 28. The second pillar 46 also comprises at least one opening 47 configured for the passage of a fluid but configured to block the passage of the oocyte. The size of the openings 47 may be comprised between 0.2 and 0.5 mm.

The flow inlet 21, flow outlet 24, oocyte inlet 22 and oocyte outlet 23 may be closed using closing devices such as sticks in the size of the entrances made of polymer such as PTFE.

The microfluidic device 100 also includes a restriction section 50 localized between the oocyte inlet 22 and the oocyte outlet 23. The restriction section 50 has a total length 32 comprised between 0.2 mm and 1 mm, preferably 0.8 mm.

As illustrated in FIGS. 2, 3 and 4, the cross-section is also a polygon. The cross-section may be a rectangle, a square, a pentagon, a hexagon and even a circle. The reduction of the cross-section may be operated along the y-direction and the z-direction, said y-direction and z-direction being orthogonal directions. Advantageously, the cross-section is equally reduced in the y-direction and the z-direction.

The restriction may be operated by a steep variation. Alternatively, the restriction section 50 may successively comprise a first ramped section 52 configured to gradually constrict the oocyte, a section of minimal dimensions 53, and a second ramped section 54 configured to gradually release restriction on the oocyte.

The length of the first ramped section 52 and of the second ramped section 54 may be comprised between 0.1 mm and 0.7 mm, preferably 0.3 mm. The section of minimal dimensions 53 may have at least one transversal dimension 34, 38, for instance either in the y direction or in the z-direction, comprised between 0.05 mm and 0.1 mm, preferably 0.07 mm. The length 31 of the section of minimal dimensions 53 may be comprised between 0.05 mm and 0.5 mm, preferably 0.2 mm.

Advantageously, the cross-section may vary in size. For instance, walls of the restriction section may be deformable and an inflatable bladder may be inserted around the restriction section 50 to induce variation in size.

### System

According to another aspect, the invention relates to a system 1000 for measuring at least one biomarker of at least one oocyte comprising the microfluidic device 100 as described above.

As illustrated in FIG. 6, the system 1000 includes a pressure controller 62. The pressure controller 62 may be configured to apply a pressure comprised between 0 and at least 350 mbar with an accuracy of 0.1 mbar.

The pressure controller 62 may be connected to an external pressure source such as a pump 61. The pump may be configured to deliver a pressure up to 1 bar depending on the pressure controller 62 used. The pressure controller 62 may also be connected to a pressurized tube 65 filled with fluid. The pressurized tube 65 is a reservoir configured for providing a flow of fluid to the microfluidic device 100. The pressurized tube 65 may be an Eppendorf tube©, or a Falcon tube© or a bottle containing culture medium, which is then smoothly and quasi-instantly injected into a microfluidic device 100. To pressurize the tube 65, the pressure controller 62 regulates pressure from the pump 61 to push gas on the fluid surface. The fluid will then flow out of the pressurized tube 65 toward the microfluidic device 100. Thus, controlling the pressure inside the pressurized tube 65 also allows to control the flow of liquid inside the microfluidic device 100 out of the tank. Advantageously, the pressure controller 62 includes a pressure sensor 67 configured to measure the pressure applied inside the microfluidic device 100. The pressure sensor 67 allows to implement a pressure regulation. The pressure controller 62 is able to regulate flow within 40 ms with a 0.005% stability. Advantageously, the pressure sensor 67 is piezoelectric-based.

The pressurized tube 65 is connected to the flow inlet 21 of the microfluidic device 100. The flow outlet 24 of the microfluidic device 100 is connected to a flow unit 70 comprising a flow sensor 75: Alternatively, the flow unit 70 may be positioned between the pressurized tube 65 and the microfluidic device 100.

By coupling the pressure controller 62 with a flow sensor 75, it is possible to precisely regulate the flow. Advantageously, the pressure controller 62 communicates 83 with a processor 80 comprising a human-man interface, such as a tablet or a computer. A pressure value may be requested by a used through the human-man interface. The pressure controller 62 may be configured to send the pressure value measured by the pressure sensor 67 to the processor 80. The processor 80 is then configured to control the pressure controller 62 to regulate the pressure in order to reach the requested pressure. The pressure may be adjusted using a proportional-integral-derivative (PID) feedback loop based on the values measured by the pressure sensor 67.

Moreover, the processor 80 is configured to receive 81 the flow values measured by the flow sensor 75. A flow rate value may also be requested by a user through the human-man interface and the processor 80 is configured to send a request to the flow unit 70 to regulate the flow. The flow may be adjusted using a proportional-integral-derivative (PID) feedback loop based on the values measured by the flow sensor 75.

The flow unit 70 is connected to a waste tube 66 configured for receiving the fluid that transited through the microfluidic device 100.

The connection between the different elements of the system may be provided by flexible tubing configured to resist to pressures comprised between 0 and 1 bar. For instance, the flexible tubing may be made of polyvinyl chloride (PVC) or polytetrafluoroethylene (PTFE).

The pressurized tube 65 and waste tube 66 may be mounted on vertical stages 63, 64 that allow to modify the height of the pressurized tube 65 or the waste tube 66. The vertical stages 63, 64 may be used to set height difference 74 between the pressurized tube 65 and the waste tube 66.

Alternatively, the pressure controller 62 may be a push-pull pressure controller connected at the inlet of the microchannel 43 via a pressurized tube 65. The push-pull pressure controller is configured to inject and aspirate fluid in and out of the microfluidic device 100. A push-pull pressure controller does not require to adjust the pressurized tube 65 and the waste tube 66 height.

Overall, the pressure controller 62 may be any pressure and/or flow generator associated with at least one sensor configured to induce sufficient fluid flow to drive the oocyte from the oocyte inlet to the oocyte outlet and configured to directly or indirectly measure a value of pressure applied into the microfluidic device 100 during the oocyte passage into the restriction section 50. Possible alternative configurations may include a flow controller combined with a pressure sensor connected to the flow inlet 21 or, a flow controller connected to the flow inlet 21 and a flow sensor in a derivative channel of known resistance.

The system 1000 also comprises an imaging device 73. The imaging device 73 may be any optical imaging device such as a standard inverted microscope coupled to a camera. The microfluidic device 100 may be positioned on the stage of the inverted microscope. Imaging may be performed at the bottom of the microfluidic device 100. Advantageously, the system 1000 includes a heating stage configured for regulating the temperature inside the microfluidic device 100.

The imaging device 73 is connected 82 to the processor 80 and configured to send the recorded images to the processor 80. For instance, the processor 80 may be connected to the camera coupled with the microscope.

The processor 80 may be configured to synchronize the recording of the images and the pressure control.

An alignment mark 57 may be drawn or etched in the microfluidic device 100.

### Method

According to another aspect, the invention relates to a method for measuring at least one biomarker of at least one oocyte using a system 1000 such as described above.

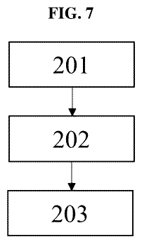

As illustrated in FIG. 7, the microfluidic device 100 is maintained at 37° C. throughout the whole measurement process and filled with a culture medium appropriate for oocytes.

The flow inlet 21 is connected to the pressurized tube 65 filled with culture medium.

The oocyte inlet 22 and the oocyte outlet 23 are closed after being completely filled with medium. The flow outlet 24 is connected in series to a flow unit 70 comprising a flow sensor 75 and to a waste tube 66 filled also with culture medium. The pressurized tube 65 is set at a lower height than the waste tube 66, so that the flow inlet 21 pressure required for no flow in the microfluidic device 100 is greater than 0 mbar. Usually, it may be set at 3 mbar. The height difference between the pressurized tube 65 and the waste tube 66 advantageously remains the same throughout the experiment.

Before oocyte deposition, the pressurized tube 65 and waste tube 66 are moved to a lower height than the microfluidic device 100 height to induce moderate suction when the oocyte inlet 21 is opened.

An oocyte is deposited in the oocyte inlet 22. The oocyte may be manually introduced using a pipette such as standard pipettes used for ART. Alternatively, it may be deposited using an automated device such as an automated pipetting robot. The oocyte may be deposited with a maximum volume of medium of 5 μl. The moderate suction helps the oocyte to sink in the oocyte inlet 22 toward the microchannel 43. The oocyte inlet 22 is then closed as well as the oocyte outlet 24 if it wasn't already closed.

Pressure is then applied 201 in the microfluidic channel 43 using the pressure controller 62 so that the oocyte is driven toward the restriction section 50. Advantageously, the oocyte is driven toward the restriction section 50 with a flow rate of about 10 μl/min. Any debris smaller than the oocyte should pass through the restriction section 50 before the oocyte and larger debris should remain blocked in the oocyte inlet 22. Once the oocyte has approached the restriction section 50, the flow rate is reduced and set to 0 μL/min to trap the oocyte without deformation. Once the oocyte has reached the restriction section 50 entrance, the pressure may be progressively increased, for instance by 0.1 mbar every 2.5 seconds to force the oocyte through the restriction section 50. Pressure and flow rate may be recorded every 50 ms for the duration of the oocyte passage.

After the oocyte exits from the restriction section 50, a flow of 10 μl/min is maintained to drive the oocyte toward the oocyte outlet 27. The pressurized tube 65 and the waste tube 66 are placed at a height above the microfluidic device 100 to induce a moderate backflow when the oocyte outlet 27 is opened. The oocyte is retrieved from the oocyte outlet 27 by pipetting with a minimum volume of 10 μl. The oocyte outlet 27 is then closed again. Flushing with a flow rate higher than 50 μl/min can be performed in case debris have entered the microfluidic channel 50 during oocyte deposition and/or retrieval.

During the passage of the oocyte in the restriction section 50, at least one image of the oocyte is acquired 202 throughout its passage in the restriction section 50. Based on these images, it is possible to measure at least one biomarker 203. Such biomarkers may include a position along the restriction section 50, a length of the oocyte or an internal flow of the oocyte.

Advantageously, an image of the oocyte may be taken every 50 ms with a resolution of around 85k DPI for the duration of the oocyte passage using an imaging device 73. Simultaneously, the flow rate and/or the pressure may be measured using the flow sensor 75 and the pressure sensor 67. Alternatively, the electric impedance may be measured in order to detect oocyte passage in the restriction section 50.

A biomarker is a defined characteristic that is measured as an indicator of normal biological processes, pathogenic processes or responses to an exposure or intervention according the FDA-NIH biomarker working group. In the case of oocyte or zygotes evaluation, a variety of different biomarkers may be measured. For instance, the biomarker may be a mechanical property of the oocyte.

The mechanical properties of an object can be deduced from force-strain response curves. In the invention, the force corresponds to the pressure or the flow and the strain can correspond to any quantitative shape parameter of the oocyte. For example, it can be defined as, but not limited to:
the percentage change in oocyte length,
the ratio of oocyte length to width, and/or
a shape descriptor such as circularity or aspect ratio.

The characteristics of the force-strain response curves at different stages of oocyte deformation may be linked to its mechanical properties. For instance, the force required for complete entry of the oocyte is linked to the oocyte cortical tension and/or elasticity. The deformation rate of the oocyte during the entry phase corresponds to the oocyte viscosity. These values may be used alone or in combination to define an oocyte mechanical score correlated with its quality.

In use, such curves may be plotted using the at least one processor 80. Alternatively, the physician may only measure the shape parameter and deduce the quality of the oocyte from abacuses.

The oocyte mechanical score can be used to guide patient management during assisted reproduction procedures, to optimize breeding farm and to promote the reproduction of endangered species.

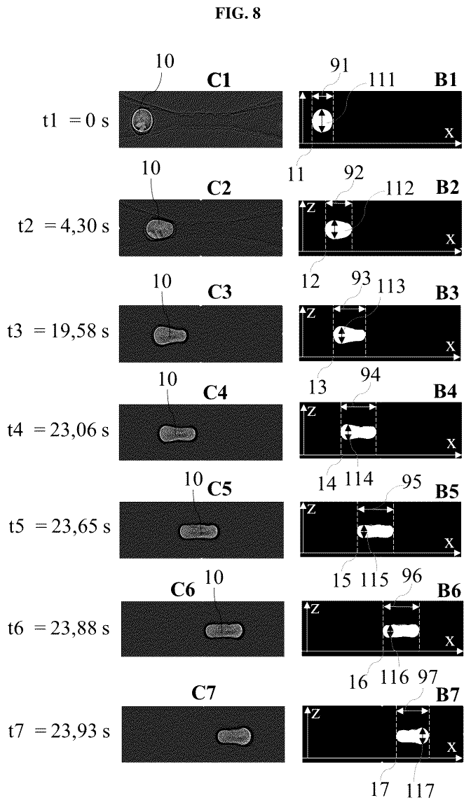

FIG. 8 is an example of images C1-C7 of the oocyte 10 taken at different time points t1-t7. The images C1-C7 show the deformations of the oocyte 10 as it passes through the restriction section 50. The recorded images C1-C7 may be analyzed to extract the position 11-17 along the x-axis and the width 91-97 of the oocyte. The position may be measured at the rear of the oocyte or at the front.

The analysis may be performed by processing the captured images C1-C7 to segment the oocyte. For instance, the captured images C1-C7 may undergo thresholding to transform them into binary images B1-B7. As a result, black and white images B1-B7 are obtained, wherein the black color corresponds to the background and the white color corresponds to the oocyte. The position 11-17 along the x-axis is determined by selecting the position along the x-axis of the first white pixel encountered. The width 91-97 of the oocyte corresponds to the distance between the first white pixel encountered and the last white pixel encountered along the x-axis. The height 111-117 corresponds to the to the distance between the first white pixel encountered and the last white pixel encountered along the z-axis.

The measurements on the images may be performed manually or using an automated routine with software such as Image J.

The position 11-17 along the x-axis may be used to plot different curves.

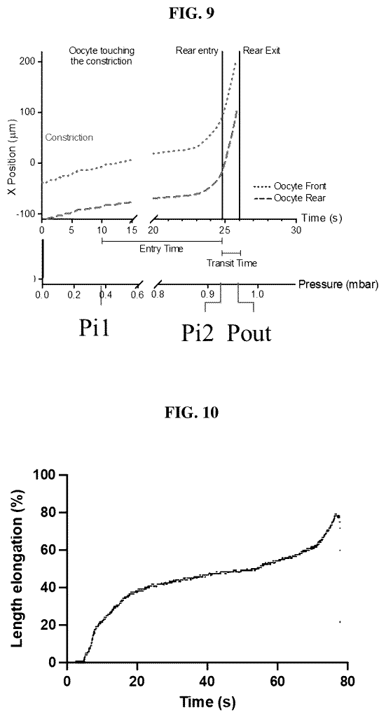

FIG. 9 is an example of such curve relating to the evolution of the position 11-17 along the x-axis as a function of time t1-t7.

The different stages of oocyte deformation such as the entry time 101 to the state of maximum deformation, the transit time 102, the exit time 103, front entry pressure Pi1, rear entry pressure Pi2 and exit pressure Pout can be established using the position of the oocyte relative to the center of the restriction section 50 and the outlet flow rate and/or pressure. The center of the restriction section 50 can easily be determined using the alignment mark 57.

The widths 91-97 may be used to determine the strain of the oocyte. The strain dynamics of the oocyte up to the maximum strain state 121 is critical to extract mechanical properties.

The strain may for instance be the length elongation of the oocyte when passing through the restriction section 50. The length elongation corresponds to the percentage of deformation when compared to the width of the undeformed oocyte, as calculated before entering the restriction section 50. In other words, as illustrated in FIG. 12, the length elongation L(t) is the ratio between the width W(t), 92-97 measured at t>0 minus the width W(t0), 91 measured before entering the restriction section 50, and the width W(t0), 91 measured before entering the restriction section 50, multiplied by 100. The length elongation L(t) may be used to plot a curve, as illustrated in FIG. 10, of the evolution of the length elongation L(t) as a function of time t1-t7.

Alternatively, the aspect ratio between the width W(t), 92-97 and the height 111-117 may be calculated and plotted a function of the pressure such as illustrated in FIG. 13.

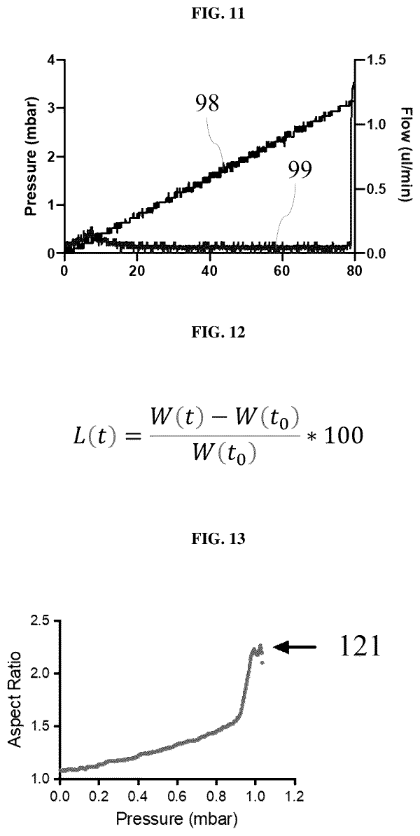

It is also possible to plot the evolution of the pressure 98 as a function of time and the evolution of the flow rate 99 as a function of time such as illustrated in FIG. 11. This curve allows to link the pressure and flow rate with time and to be able to know the pressure and flow rate applied in the microfluidic device 100 for each image. For instance, it allows to determine the front entry pressure Pi1, the rear entry pressure Pi2 and the oocyte exit pressure Pout such as illustrated in FIG. 9. Measurements of the flux also help verifying the equilibrium pressure corresponding to the oocyte completely plugging the restriction section 50.

Steps 201 and 202 can be fully automated by implementing real-time oocyte tracking and flow feedback with software such as Labwiew and Micro-Manager. Detection of an oocyte at the restriction can trigger the flow to stop and the start of pressure increase and image recording. A positive flow indicates that the oocyte has exited from the restriction and can trigger the stop of the recording and increase of the pressure.

1-11. (canceled)

12. A microfluidic device for use in a system for measuring at least one biomarker, of at least one oocyte, said microfluidic device comprising at least one microfluidic channel configured for a passage of a flow of fluid comprising at least one oocyte, the microfluidic channel comprising a restriction section configured to deform the oocyte in at least a first direction and a second direction perpendicular to a microfluidic channel axis, the first direction and the second direction being non-aligned.

13. The microfluidic device according to claim 12, wherein the oocyte has an oocyte size and the restriction section has a transversal section comprised between 70% and 90% of the oocyte size.

14. The microfluidic device according to claim 13, wherein the oocyte has an oocyte size and the restriction section has a transversal section comprised between 75% and 85% of the oocyte size.

15. The microfluidic device according to claim 4, wherein the oocyte has an oocyte size and the restriction section has a transversal section of 80% of the oocyte size.

16. The microfluidic device according to claim 12, wherein the restriction along the at least first direction is identical to the restriction along the second direction.

17. The microfluidic device according to claim 12, wherein the microfluidic channel has a polygonal-shaped cross-section.

18. The microfluidic device according to claim 12, wherein the restriction section successively comprises:
a first ramped section configured to gradually constrict the oocyte,
a section of minimal dimensions, and
a second ramped section configured to gradually release restriction on the oocyte.

19. The microfluidic device according to claim 12, wherein the restriction section comprises at least one transversal dimension that is variable.

20. The microfluidic device according to claim 12, wherein the restriction section comprises at least one wall that is deformable along at least one of the first direction and second direction.

21. A system for measuring at least one biomarker of at least one oocyte comprising:
the microfluidic device according to claim 12,
a pressure controller configured for applying a pressure on the oocyte to transport the oocyte through the restriction section, and
an imaging device configured for acquiring at least one image of the oocyte during its passage into the restriction section, and
at least one processor configured to process the at least one image to measure the at least one biomarker.

22. The system according to claim 21, wherein the processor is configured to determine a position of the oocyte.

23. The system according to claim 21, wherein the processor is configured to determine a strain of the oocyte.

24. A method for measuring at least one biomarker of at least one oocyte using a system according to claim 21, comprising:
applying a pressure into the microfluidic device, to transport at least one oocyte through the restriction section,
acquiring at least one image of the oocyte during its passage into the restriction section, and
processing the at least one image to measure the at least one biomarker.
